(19) United States
(12) Patent Application Publication
(10) Pub. No.: US 2025/0073136 A1
(43) Pub. Date: Mar. 6, 2025

(54) IMPROVEMENTS IN OR RELATING TO ORGANIC COMPOSITIONS

(71) Applicant: GIVAUDAN SA, VERNIER (CH)

(72) Inventors: Sarah EL-HABNOUNI, Singapore (SG); Mandy NG YI QIN, Singapore (SG); Lek Keat ONG, Singapore (SG)

(21) Appl. No.: 18/725,296

(22) PCT Filed: Jan. 27, 2023
(86) PCT No.: PCT/EP2023/051968

§ 371 Date: Jun. 28, 2024

(30) Foreign Application Priority Data
Jan. 31, 2022 (GB) 2201197.7

(51) Int. Cl. A61K 8/11

(52) U.S. Cl. CPC A61K 8/11; A61K 2800/52; A61K 2800/5424; A61K 2800/56; A61K 2800/80

(57) ABSTRACT

The present invention provides an encapsulated composition comprising at least one core-shell microcapsule, wherein the at least one core-shell microcapsule comprises a core comprising at least one benefit agent and a shell surrounding the core, wherein the shell comprises a network of cross-linked resin, wherein the resin comprises a terpolymer and a polymeric stabilizer, wherein the terpolymer comprises
(a) moieties derived from at least one polyamine,
(b) moieties derived from a milk protein or a milk protein derivative,
(c) moieties derived from the group consisting of alkylene and alkylenoxy moieties having 1 to 6 methylene units, preferably 1 to 4 methylene units and most preferably 1 methylene unit.

The invention also relates to a method for preparing such encapsulated compositions and to their use to enhance the performance of a benefit agent in a consumer product.

The present invention is concerned with encapsulated compositions comprising at least one core-shell microcapsule. The invention also relates to a method for preparing such encapsulated compositions and to their use to enhance the performance of a benefit agent in a consumer product.

### BACKGROUND OF THE INVENTION

It is known to incorporate encapsulated benefit agents in consumer products, such as household care, personal care, fabric care and pet care products. Benefit agents include for example fragrances, cosmetic agents, food ingredients, nutraceuticals, drugs and substrate enhancers.

Microcapsules that are particularly suitable for delivery of such benefit agents are core-shell microcapsules, wherein the core usually comprises the benefit agent and the shell is impervious or partially impervious to the benefit agent. Generally, these microcapsules are employed in aqueous media and the encapsulated benefit agents are hydrophobic. A broad selection of shell materials can be used, provided the shell material is impervious or partially impervious to the encapsulated benefit agent.

Benefit agents are encapsulated for a variety of reasons. Microcapsules can isolate and protect such materials from external suspending media, such as consumer product bases, in which they may be incompatible or unstable. They are also used to assist in the deposition of benefit agents onto substrates, such as skin or hair, fabrics or hard household surfaces in case of perfume ingredients. They can also act as a means of controlling the spatio-temporal release of a benefit agent.

In order for microcapsules to be suitable for various applications it is necessary that they are sufficiently robust (i.e. sufficiently stable and not leaky during manufacture and storage), while at the same time enabling an acceptable release profile of the core contents, as desired in each application.

Aminoplast microcapsules are among the most commonly used encapsulating media for ingredients such as fragrances, insecticides, malodour counteracting substances, fungicides and mildewicides, and the like. There are established processes of forming aminoplast microcapsules that are well documented in the prior art. Typically, in a first step an oil-in-water emulsion is formed, consisting of fragrance-containing oil droplets dispersed in an aqueous continuous phase. Thereafter, shell-forming amino-aldehyde pre-condensates contained in the emulsion are caused to form encapsulating polymeric shells around the benefit agent-containing droplets to form core-shell microcapsules. Reagents and reaction conditions are selected to ensure the amino-aldehyde pre-condensates undergo poly-condensation and crosslinking to form polymeric shells rapidly around the oil droplets, thereby retaining all, or substantially all, of the benefit agent ingredients within the droplets and preventing subsequent leakage of encapsulated benefit agent ingredients from the microcapsules. If the shells are unable to form quickly then it may be impossible to form microcapsules, or if microcapsules can be formed they may be characterized by poor benefit agent retention and may be prone to agglomeration.

For example, WO 2017/001672A1A1, WO 2016/207180A1 and WO 2008/098387A1 disclose aminoplast core-shell microcapsules. These microcapsules have excellent properties, both in manufacture and application.

However, consumers are increasingly concerned about using materials obtained from non-renewable sources, such as synthetic petrochemicals, as well as about the processes for manufacturing the consumer products. The “clean label” concept is one of the biggest trends of the decade. The term itself has many definitions including sustainable, naturally sourced and biodegradable ingredients as well as minimal processing and impact on the environment. Nevertheless, it is generally difficult to use natural materials or materials derived from nature to satisfy the requirements for suitable encapsulation compositions. Bio-based ingredients for customer formulations must provide a unique combination of performance and sustainability, so consumers feel confident in the safety and efficacy of these ingredients.

Therefore, there is still a need to provide encapsulated compositions that are sustainable and comprise increased levels of natural materials or materials derived from nature, in particular with good predicted biodegradability, whilst satisfying the required balance between robustness and benefit-agent release properties, during all stages of manufacture, storage and use. Furthermore, the processes of manufacturing the compositions should follow the “clean label” requirements, in addition to being safe, robust and cost-efficient.

### SUMMARY OF THE INVENTION

In a first aspect, the invention provides an encapsulated composition comprising at least one core-shell microcapsule, wherein the at least one core-shell microcapsule comprises a core comprising at least one benefit agent and a shell surrounding the core, wherein the shell comprises a network of cross-linked resin, wherein the resin comprises a terpolymer and a polymeric stabilizer, wherein the terpolymer comprises
(a) moieties derived from at least one polyamine,
(b) moieties derived from a milk protein or a milk protein derivative,
(c) moieties derived from the group consisting of alkylene and alkylenoxy moieties having 1 to 6 methylene units, preferably 1 to 4 methylene units and most preferably 1 methylene unit.

In a further aspect, the invention provides methods for preparing an encapsulated composition as defined hereinbefore.

In another aspect, it is provided a consumer product comprising an encapsulated composition as defined hereinbefore.

The invention further provides use of an encapsulated composition as defined hereinbefore to improve the perception or enhance the performance of the benefit agent in a consumer product.

### Definitions

The term “benefit agent” refers to any substance which, when added to a product, may improve the perception of this product by a consumer or may enhance the action of this product in an application. Examples of benefit agents include perfume ingredients, flavor ingredients, cosmetic ingredients, bioactive agents (such as bactericides, insect repellents and pheromones), substrate enhancers (such as silicones and brighteners), enzymes (such as lipases and proteases), dyes, pigments and nutraceuticals.

By “moiety” is meant a chemical entity, which is part of the terpolymer and which is derived from a particular molecule.

The term “polyamine” refers to an organic compound having more than two amino groups in the molecule.

The use of the term “derived from” does not necessarily mean that the moiety in the terpolymer is directly derived from the substance itself, although this may be (and often is) the case. In fact, one of the more convenient methods of preparing the terpolymer involves the use of alkylolated polyamines as starting materials; these combine in a single molecule both the moieties (a) and (c) mentioned hereinabove.

As is conventional in the art, a “protein” is a linear organic polymer composed of amino acid residues bonded together in a chain, forming part of (or the whole of) a protein molecule. “Protein” as used herein means a natural polypeptide, polypeptide derivative, and/or modified polypeptide. The polypeptide may exhibit an average molecular weight of from 1,000 Da to 40,000,000 Da and/or greater than 10,000 Da and/or greater than 100,000 Da and/or greater than 1,000,000 Da and/or less than 3,000,000 Da and/or less than 1,000,000 Da and/or less than 500,000 Da, or a range delimited by any one of these molecular weights.

The term “bio-based” relates to the origin of a material and refers to materials intentionally made from substances derived from living (or once-living) organisms, as opposed to petroleum-derived materials. The definition includes both natural materials, such as naturally-extracted proteins and polysaccharides, and materials that have undergone some degree of processing, such as cellulose fibers.

“Biodegradable” materials are defined as materials whose physical and chemical properties undergo deterioration and completely degrade when exposed to the environment. This property, therefore, relates to the end-of-life of the material. Bio-based materials can be biodegradable or non-degradable. Similarly, while many bio-based materials are biodegradable (e.g., starch), not all biodegradable materials are bio-based.

In context of the present invention, a “biodegradable” ingredient, or a “biodegradable” material in general, for instance a shell material, is a material which meets the pass criteria for “inherently biodegradable” and/or “readily biodegradable” in at least one OECD biodegradation study. In order to avoid any ambiguity, this means that if an ingredient passes one test but fails one or more other ones, the pass result overrules the other test results.

For assessment of the pass criteria for “readily biodegradable”, the biodegradation study can be selected from the group consisting of OECD Method 301B, OECD Method 301C, OECD Method 301D, OECD Method 301F and OECD Method 310.

OECD Method 301B, OECD Method 301C, OECD Method 301D and OECD Method 301F are described in the OECD Guidelines for the Testing of Chemicals, Section 3, Test No. 301: Ready Biodegradability (Adopted: 17th July 1992; https://doi.org/10.1787/97892640703-(9-en).

OECD Method 310 is described in the OECD Guidelines for the Testing of Chemicals, Section 3, Test No. 310: Ready Biodegradability—CO2 in sealed vessels (Headspace Test) (Adopted: 23 Mar. 2006; Corrected: 26 September 2014; https://doi.org/10.1787/9789264016316-en).

In a particular aspect of the present invention, the pass criteria for “readily biodegradable” are assessed according to OECD Method 301F, which refers to manometric respirometry. In this method the pass level for “ready biodegradability” is to reach 60% of theoretical oxygen demand and/or chemical oxygen demand. This pass value has to be reached in a 10-day window within the 28-day period of the test. The 10-day window begins when the degree of biodegradation has reached 10% of theoretical oxygen demand and/or chemical oxygen demand and must end before day 28 of the test.

Given a positive result in a test of ready biodegradability, it may be assumed that the chemical will undergo rapid and ultimate biodegradation in the environment (Introduction to the OECD Guidelines for the Testing of Chemicals, Section 3, Part 1: Principles and Strategies Related to the Testing of Degradation of Organic Chemicals; Adopted: July 2003).

For assessment of the pass criteria for “inherently biodegradable”, the biodegradation study can be OECD Method 302C, but also OECD Method 301F can be used, although with different pass criteria. Also these methods are suitable for volatile materials.

OECD Method 302C is described in the OECD Guidelines for the Testing of Chemicals, Section 3, Test No. 302C: Inherent Biodegradability: Modified MITI Test (II) (Adopted: 12 May 1981; Corrected 8 Sep. 2009; https://doi.org/10.1787/9789264070400-en).

In a particular aspect of the present invention, the pass criteria for “inherently biodegradable” are assessed by OECD Method 302C. In this method the pass level for “inherently biodegradability” is then to reach 70% of theoretical oxygen demand. There is no time limit to reach this level.

Biodegradation rates above 70% may be regarded as evidence of inherent, ultimate biodegradability (OECD Guidelines for the Testing of Chemicals, Section 3, Part 1: Principles and Strategies Related to the Testing of Degradation of Organic Chemicals; Adopted: July 2003).

If OECD Method 301F is used for assessment of the pass criteria for “inherently biodegradable”, the pass level is 60% of theoretical oxygen demand and/or chemical oxygen demand. This pass value can be reached after the 28-day period of the test, which is usually extended to 60 days. No 10-day window applies.

In the present context, if an ingredient is an essential oil, it is considered to be a “biodegradable ingredient” if all of its constituents present at a level ≥1% fall under the definition of “inherently biodegradable” and/or “readily biodegradable” as defined herein above. However, the essential oil can also be subjected to the above-mentioned biodegradation tests.

In the context of the present invention, the leakage is considered as significantly reduced if the amount of the benefit agent that has leached in a consumer product base within a period of 1 month at 37° C. is less than 75%, preferably less than 50%, more preferably less than 25%, and still more preferably less than 10% of the nominal amount of encapsulated benefit agent.

In the context of the present invention, all percentages refer to weight percentages (% w/w), unless otherwise indicated.

### DETAILED DESCRIPTION

Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

### Encapsulated Composition

The applicant has surprisingly and unexpectedly found that an encapsulated composition comprising at least one core-shell microcapsule, wherein the at least one core-shell microcapsule comprises a core comprising at least one benefit agent and a shell surrounding the core, wherein the shell comprises a network of cross-linked resin, wherein the resin comprises a terpolymer and a polymeric stabilizer, wherein the terpolymer comprises
(a) moieties derived from at least one polyamine,
(b) moieties derived from a milk protein or a milk protein derivative,
(c) moieties derived from the group consisting of alkylene and alkylenoxy moieties having 1 to 6 methylene units, preferably 1 to 4 methylene units and most preferably 1 methylene unit
satisfies both the performance requirements (i.e. displays a suitable balance between robustness and benefit-agent release properties, during all stages of manufacture, storage and use) and is biodegradable.

The novel use of a milk protein or a milk protein derivative in the composition of the terpolymer led to the surprising advantage that the microcapsules formed were biodegradable, but retained the stability and performance characteristics that were comparable with those of the microcapsules cross-linked with resorcinol. The microcapsules formed were white, unlike microcapsules made using resorcinol. The use of a milk protein or a milk protein derivative in this way provides a shell that has a novel structure and has advantages over prior art aminoplast capsules.

The invention, therefore, provides an encapsulated composition comprising at least one core-shell microcapsule, wherein the at least one core-shell microcapsule comprises a core comprising at least one benefit agent and a shell surrounding the core, wherein the shell comprises a network of cross-linked resin, wherein the resin comprises a terpolymer and a polymeric stabilizer, wherein the terpolymer comprises
(a) moieties derived from at least one polyamine,
(b) moieties derived from a milk protein or a milk protein derivative,
(c) moieties derived from the group consisting of alkylene and alkylenoxy moieties having 1 to 6 methylene units, preferably 1 to 4 methylene units and most preferably 1 methylene unit.

In one embodiment, the encapsulated composition is an encapsulated perfume composition.

### Benefit Agent

Suitable benefit agents to be incorporated into the core of the core-shell microcapsules of the present invention include perfume or fragrance ingredients, flavor ingredients, cosmetic ingredients, bioactive agents (such as bactericides, insect repellents and pheromones), substrate enhancers (such as silicones and brighteners), enzymes (such as lipases and proteases), dyes, pigments and nutraceuticals

In one embodiment, the at least one benefit agent may be at least one fragrance ingredient. A comprehensive list of fragrance ingredients that may be encapsulated in accordance with the present invention may be found in the perfumery literature, for example “Perfume & Flavor Chemicals”, S. Arctander (Allured Publishing, 1994). Encapsulated fragrance ingredients according to the present invention preferably comprise fragrance ingredients selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER CORE1-((2-(tert-butyl)cyclohexyl)oxy) butan-2-olAMBERKETAL (3,8,8,11a-tetramethyldodecahydro-1H-3,5a-epoxynaphtho[2,1-c]oxepine); AMBERMAX (1,3,4,5,6,7-hexahydro-.beta., 1,1,5,5-pentamethyl-2H-2,4a-Methanonaphthalene-8-ethanol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYL SALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-BORNEOL methylenebicyclo[3.3.1]nonane); CRYSTALS ((1S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl) propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl) quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4 (5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl) hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecanenitrile); CORANOL (4-cyclohexyl-2-methylbutan-2-ol); COSMONE ((Z)-3-methylcyclotetradec-5-enone); COUMARIN PURE CRYSTALS S (2H-chromen-2-one); CRESYL ACETATE PARA ((4-methylphenyl) acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); CYMENE PARA (1-methyl-4-propan-2-ylbenzene); ((E)-1-(2,6,6-trimethylcyclohexa-1,3-dien-1-yl) but-2-en-1-one); DAMASCENONE DAMASCONE ALPHA ((E)-1-(2,6,6-trimethylcyclohex-2-en-1-yl) but-2-en-1-one); DAMASCONE DELTA (1-(2,6,6-trimethyl-1-cyclohex-3-enyl) but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE (3-methyl-2-pentylcyclopent-2-enone); DIHYDRO MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5 (6H)-ylidene) butanal); EBANOL ((E)-3-methyl-5-(2,2,3-trimethylcyclopent-3-en-1-yl) pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl) ethanone); FLORALOZONE (3-(4-ethylphenyl)-2,2-dimethylpropanal); FLORHYDRAL (3-(3-isopropylphenyl) butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl) pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HELIOTROPINE CRYSTALS (benzo[d][1,3]dioxole-5-carbaldehyde); HERBANATE ((2S)-ethyl 3-isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (1H-indole); INDOLENE (8,8-di(1H-indol-3-yl)-2,6-dimethyloctan-2-ol); IONONE BETA ((E)-4-(2,6,6-trimethylcyclohex-1-en-1-yl) but-3-en-2-one); IRISANTHEME ((E)-3-methyl-4-(2,6,6-trimethylcyclohex-2-en-1-yl) but-3-en-2-one); IRISONE ALPHA ((E)-4-(2,6,6-trimethylcyclohex-2-en-1-yl) but-3-en-2-one); IRONE ALPHA ((E)-4-(2,5,6,6-tetramethylcyclohex-2-en-1-yl) but-3-en-2-one); ISO E SUPER (1-(2,3,8,8-tetramethyl-1,2,3,4,5,6,7,8-octahydronaphthalen-2-yl) ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl) phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl) but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl) methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy) hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); #N/ALINALOOL (3,7-dimethylocta-1,6-dien-3-ol); LINALOOL OXIDE (2-(5-methyl-5-vinyltetrahydrofuran-2-yl) propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl) methanol); MEFROSOL (3-methyl-5-phenylpentan-1-ol); MELONAL (2,6-dimethylhept-5-enal); #N/A #N/AMERCAPTO-8-METHANE-3-ONE (mercapto-para-menthan-3-one); METHYL ANTHRANILATE (methyl 2-aminobenzoate); METHYL BENZOATE (methyl benzoate); METHYL CEDRYL KETONE (1-((1S,8aS)-1,4,4,6-tetramethyl-2,3,3a,4,5,8-hexahydro-1H-5,8a-methanoazulen-7-yl) ethanone); METHYL CINNAMATE (methyl 3-phenylprop-2-enoate); METHYL DIANTILIS (2-ethoxy-4-(methoxymethyl) phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl) propyl)cyclopentanone); NEOBERGAMATE FORTE (2-methyl-6-methyleneoct-7-en-2-yl acetate); NEOCASPIRENE EXTRA (10-isopropyl-2,7-dimethyl-1-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7,11-trimethyldodeca-1,6,10-trien-3-yl acetate); NEROLINE CRYSTALS (2-ethoxynaphthalene); NEROLIONE (1-(3-methylbenzofuran-2-yl) ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclo[3.1.1]hept-2-en-2-yl)ethyl acetate); NYMPHEAL (3-(4-(2-methylpropyl)-2-methylphenyl) propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl) butanamide); PEACH PURE (5-heptyldihydrofuran-2 (3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl) acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-trimethylbicyclo[3.1.1]hept-2-ene); PINENE BETA (6,6-dimethyl-2-methylenebicyclo[3.1.1]heptane); PINOACETALDEHYDE (3-(6,6-dimethylbicyclo[3.1.1]hept-2-en-2-yl) propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2 (3H)-one); RADJANOL SUPER ((E)-2-ethyl-4-(2,2,3-trimethylcyclopent-3-en-1-yl) but-2-en-1-ol); RASPBERRY KETONE (4-(4-hydroxyphenyl) butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE EXTRA (3-methyl-5-(2,2,3-trimethylcyclopent-3-en-1-); SCENTAURUS CLEAN (ethyl (Z)-2-acetyl-4-methyltridec-2-enoate); yl) pentan-2-ol); SCENTAURUS JUICY (4-(dodecylthio)-4-methylpentan-2-one); SERENOLIDE (2-(1-(3,3-dimethylcyclohexyl) ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl) pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl) propan-2-ol); TERPINEOL ALPHA (2-(4-methyl-1-cyclohex-3-enyl) propan-2-ol); TERPINEOL PURE (2-(4-methylcyclohex-3-en-1-yl) propan-2-ol); TERPINOLENE (1-methyl-4-(propan-2-ylidene)cyclohex-1-ene); TERPINYL ACETATE (2-(4-methyl-1-cyclohex-3-enyl) propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

In particularly preferred embodiments of the present invention, more than 75%, preferably more than 80%, even more preferably more than 85%, even still more preferably more than 90%, even yet still more preferably more than 95%, of the fragrance ingredients are biodegradable and selected from ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA (2-methylundecanal); ALDEHYDE C 8 OCTYLIC (octanal); CYCLAMEN ALDEHYDE EXTRA (3-(4-isopropylphenyl)-2-methylpropanal); ALDEHYDE ISO C 11 ((E)-undec-9-enal); ALLYL AMYL GLYCOLATE (prop-2-enyl 2-(3-methylbutoxy)acetate); ALLYL CYCLOHEXYL PROPIONATE (prop-2-enyl 3-cyclohexylpropanoate); ALLYL OENANTHATE (prop-2-enyl heptanoate); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL SALICYLATE (pentyl 2-hydroxybenzoate); AUBEPINE PARA CRESOL (4-methoxybenzaldehyde); BENZYL ACETATE (benzyl acetate); BENZYL SALICYLATE (benzyl 2-hydroxybenzoate); BORNYL ACETATE ((2S,4S)-1,7,7-trimethylbicyclo[2.2.1]heptan-2-yl acetate); CARVACROL (5-isopropyl-2-methylphenol); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CITRAL ((E)-3,7-dimethylocta-2,6-dienal); CITRONELLOL (3,7-dimethyloct-6-en-1-ol); CITRONELLYL ACETATE (3,7-dimethyloct-6-en-1-yl acetate); COSMONE ((Z)-3-methylcyclotetradec-5-enone); CRESYL METHYL ETHER PARA (1-methoxy-4-methylbenzene); CYCLOHEXYL ETHYL ACETATE (2-cyclohexylethyl acetate); CYCLOHEXYL SALICYLATE (cyclohexyl 2-hydroxybenzoate); DAMASCENONE ((E)-1-(2,6,6-trimethylcyclohexa-1,3-dien-1-yl) but-2-en-1-one); DAMASCONE ALPHA ((E)-1-(2,6,6-trimethylcyclohex-2-en-1-yl) but-2-en-1-one); DECALACTONE GAMMA (5-hexyloxolan-2-one); DECENAL-4-TRANS ((E)-dec-4-enal); DIHYDRO MYRCENOL (2,6-dimethyloct-7-en-2-ol); DIPHENYL OXIDE (oxydibenzene); DIHYDRO ANETHOLE (1-methoxy-4-propylbenzene); DIHYDRO JASMONE (3-methyl-2-pentylcyclopent-2-enone); DIMETHYL ANTHRANILATE (methyl 2-(methylamino)benzoate); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETOL (2,6-dimethylheptan-2-ol); DODECALACTONE DELTA (6-heptyltetrahydro-2H-pyran-2-one); DODECALACTONE GAMMA (5-octyloxolan-2-one); DODECENAL ((E)-dodec-2-enal); EBANOL ((E)-3-methyl-5-(2,2,3-trimethylcyclopent-3-en-1-yl) pent-4-en-2-ol); ETHYL HEXANOATE (ethyl hexanoate); ETHYL METHYL-2-BUTYRATE (ethyl 2-methyl butyrate); ETHYL MALTOL (2-ethyl-3-hydroxy-4H-pyran-4-one); ETHYL OENANTHATE (ethyl heptanoate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FIXAMBRENE (3a,6,6,9a-tetramethyldodecahydronaphtho[2,1-b]furan); FLORHYDRAL (3-(3-isopropylphenyl) butanal); FLORIDILE ((E)-undec-9-enenitrile); GALBANONE PURE (1-(5,5-dimethylcyclohex-1-en-1-yl) pent-4-en-1-one); GARDENOL (1-phenylethyl acetate); GERANIOL ((E)-3,7-dimethylocta-2,6-dien-1-ol); GERANYL ACETATE ((E)-3,7-dimethylocta-2,6-dien-1-yl acetate); HABANOLIDE ((E)-oxacyclohexadec-12-en-2-one); HEDIONE (methyl 3-oxo-2-pentylcyclopentaneacetate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS SALICYLATE ((Z)-hex-3-en-1-yl 2-hydroxybenzoate); HEXYL ACETATE (hexyl acetate); INDOLENE (8,8-di(1H-indol-3-yl)-2,6-dimethyloctan-2-ol); IONONE BETA ((E)-4-(2,6,6-trimethylcyclohex-1-en-1-yl) but-3-en-2-one); IRISANTHEME ((E)-3-methyl-4-(2,6,6-trimethylcyclohex-2-en-1-yl) but-3-en-2-one); IRISONE ALPHA ((E)-4-(2,6,6-trimethylcyclohex-2-en-1-yl) but-3-en-2-one); ISOAMYL ACETATE (3-methylbutyl acetate); ISOAMYL BUTYRATE (3-methylbutyl butanoate); ISOEUGENOL ((E)-2-methoxy-4-(prop-1-en-1-yl) phenol); ISOJASMONE B 11 (2-hexylcyclopent-2-en-1-one); ISORALDEINE ((E)-3-methyl-4-(2,6,6-trimethylcyclohex-2-en-1-yl) but-3-en-2-one); JASMONYL (3-butyl-5-methyltetrahydro-2H-pyran-4-yl acetate); LAITONE (8-isopropyl-1-oxaspiro[4.5]decan-2-one); LEMONILE ((2E,6Z)-3,7-dimethylnona-2,6-dienenitrile); LINALOOL (3,7-dimethylocta-1,6-dien-3-ol); LINALOOL OXIDE (2-(5-methyl-5-vinyltetrahydrofuran-2-yl) propan-2-ol); LINALYL ACETATE (3,7-dimethylocta-1,6-dien-3-yl acetate); MANZANATE (ethyl 2-methylpentanoate); MAYOL ((4-isopropylcyclohexyl) methanol); MEFROSOL (3-methyl-5-phenylpentan-1-ol); MELONAL (2,6-dimethylhept-5-enal); MERCAPTO-8-METHANE-3-ONE (mercapto-para-menthan-3-one); METHYL ANTHRANILATE (methyl 2-aminobenzoate); METHYL BENZOATE (methyl benzoate); METHYL DIANTILIS (2-ethoxy-4-(methoxymethyl) phenol); METHYL HEPTENONE PURE (6-methylhept-5-en-2-one); METHYL LAITONE (8-methyl-1-oxaspiro[4.5]decan-2-one); METHYL OCTYNE CARBONATE (methyl non-2-ynoate); METHYL SALICYLATE (methyl 2-hydroxybenzoate); NECTARYL (2-(2-(4-methylcyclohex-3-en-1-yl) propyl)cyclopentanone); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLINE CRYSTALS (2-ethoxynaphthalene); NEROLIONE (1-(3-methylbenzofuran-2-yl) ethanone); NERYL ACETATE ((Z)-3,7-dimethylocta-2,6-dien-1-yl acetate); NONADIENAL ((2E,6Z)-nona-2,6-dienal); NONENAL-6-CIS ((Z)-non-6-enal); NONENOL-6-CIS ((Z)-non-6-en-1-ol); NYMPHEAL (3-(4-(2-methylpropyl)-2-methylphenyl) propanal); OCTALACTONE DELTA (6-propyltetrahydro-2H-pyran-2-one); ORANGER CRYSTALS (1-(2-naphtalenyl)-ethanone); PARA TERT BUTYL CYCLOHEXYL ACETATE (4-(tert-butyl)cyclohexyl acetate); PEACH PURE (5-heptyldihydrofuran-2 (3H)-one); PELARGOL (3,7-dimethyloctan-1-ol); PHENYL ETHYL ACETATE (2-phenylethyl acetate); PINENE ALPHA (2,6,6-trimethylbicyclo[3.1.1]hept-2-ene); PINENE BETA (6,6-dimethyl-2-methylenebicyclo[3.1.1]heptane); POMAROSE ((2E,5E)-5,6,7-trimethylocta-2,5-dien-4-one); POMELOL FF (2,4,7-Trimethyl-6-octen-1-ol); PRENYL ACETATE (3-methylbut-2-en-1-yl acetate); PRUNOLIDE (5-pentyldihydrofuran-2 (3H)-one); RASPBERRY KETONE (4-(4-hydroxyphenyl) butan-2-one); ROSALVA (dec-9-en-1-ol); ROSE OXIDE CO (4-methyl-2-(2-methylprop-1-en-1-yl)tetrahydro-2H-pyran); ROSYRANE SUPER (4-methyl-2-phenyl-3,6-dihydro-2H-pyran); SAFRANAL (2,6,6-trimethylcyclohexa-1,3-dienecarbaldehyde); SCENTAURUS JUICY (4-(dodecylthio)-4-methylpentan-2-one); SILVIAL (2-methyl-3-[4-(2-methylpropyl)phenyl]propanal); STYRALLYL ACETATE (1-phenylethyl acetate); SYLKOLIDE ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINEOL (2-(4-methylcyclohex-3-en-1-yl) propan-2-ol); TERPINOLENE (1-methyl-4-(propan-2-ylidene)cyclohex-1-ene); TETRAHYDRO LINALOOL (3,7-dimethyloctan-3-ol); TOSCANOL (1-(cyclopropylmethyl)-4-methoxybenzene); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); UNDECAVERTOL ((E)-4-methyldec-3-en-5-ol); YARA YARA (2-methoxynaphtalene); BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These ingredients have the advantage of providing microcapsules which are particularly sustainable.

The at least one benefit agent may comprise at least one fragrance precursor, meaning a material that is capable of releasing a fragrance ingredient by the means of a stimulus, such as a change of temperature, the presence of oxidants, the action of enzymes or the action of light. Such fragrance precursors are well-known to the art.

The at least one benefit agent may also comprise at least one functional cosmetic ingredient. The functional cosmetic ingredients for use in the encapsulated composition are preferably hydrophobic. Preferably, the cosmetic ingredients have a calculated octanol/water partition coefficient (ClogP) of 1.5 or more, more preferably 3 or more. Alternatively preferred, the ClogP of the cosmetic ingredient is from 2 to 7.

Particularly useful functional cosmetic ingredients may be selected from the group consisting of emollients, smoothening ingredients, hydrating ingredients, soothing and relaxing ingredients, decorative ingredients, deodorants, anti-aging ingredients, cell rejuvenating ingredients, draining ingredients, remodeling ingredients, skin levelling ingredients, preservatives, anti-oxidants, antibacterial or bacteriostatic ingredients, cleansing ingredients, lubricating ingredients, structuring ingredients, hair conditioning ingredients, whitening ingredients, texturing ingredients, softening ingredients, anti-dandruff ingredients, and exfoliating ingredients.

Particularly useful functional cosmetic ingredients include, but are not limited to hydrophobic polymers, such as alkyldimethylsiloxanes, polymethylsil-sesquioxanes, polyethylene, polyisobutylene, styrene-ethylene-styrene and styrene-butylene-styrene block copolymers, and the like; mineral oils, such as hydrogenated isoparaffins, silicone oils and the like; vegetable oils, such as argan oil, jojoba oil, aloe vera oil, and the like; fatty acids and fatty alcohols and their esters; glycolipides; phospholipides; sphingolipides, such as ceramides; sterols and steroids; terpenes, sesquiterpenes, triterpenes and their derivatives; essential oils, such as Arnica oil, Artemisia oil, Bark tree oil, Birch leaf oil, Calendula oil, Cinnamon oil, Echinacea oil, Eucalyptus oil, Ginseng oil, Jujube oil, Helianthus oil, Jasmine oil, Lavender oil, Lotus seed oil, Perilla oil, Rosmary oil, Sandal wood oil, Tea tree oil, Thyme oil, Valerian oil, Wormwood oil, Ylang Ylang oil, and Yucca oil.

In particular, the at least one functional cosmetic ingredient may be selected from the group consisting of Sandal wood oil, such as Fusanus Spicatus kernel oil; Panthenyl triacetate; Tocopheryl acetate; Tocopherol; Naringinin; Ethyl linoleate; Farnesyl acetate; Farnesol; Citronellyl methyl crotonate; and Ceramide-2 (1-Stearoiyl-C18-Sphingosine, CAS-No: 100403-19-8).

The at least one benefit agent may comprise agents which suppress or reduce malodour and its perception by adsorbing odour, agents which provide a warming or cooling effect, insect repellents or UV absorbers.

### Terpolymer

The cross-linked resin is formed when an amino-aldehyde pre-condensate is caused to undergo a poly-condensation reaction to form the resin, and the milk protein or milk protein derivative cross-links with the poly-condensed material to form the network of cross-linked resin.

The polyamine-aldehyde pre-condensate may be any polyamine-aldehyde pre-condensate comprising the moieties described herein, and it may be prepared by any of the many suitable methods known to the art.

Example of suitable polyamine moieties include, but are not limited to, those derived from melamine and glycouril.

Amino-aldehyde pre-condensates useful in the preparation of thermoplast resins are well known in the art. Suitable amino-aldehyde pre-condensates include but are not limited to partially methylated mono- and poly-methylol-2,4,6-triamino-1,3,5-triazine pre-condensates, such as those commercially available under the Trade Mark CYMEL (ex Cytec Technology Corp.) or LURACOLL (ex BASF), and/or mono- and polyalkylol-glycouril pre-condensates. These alkylolated polyamines may be provided in partially alkylated forms, obtained by addition of short chain alcohols having typically 1 to 6 methylene units. These partially alkylated forms are known to be less reactive and therefore more stable during storage. Preferred polyalkylol-polyamines are polymethylol-melamines.

Alternatively, poly[N-(2,2-dimethoxy-1-hydroxy)] polyamines can be used, including tri-[N-(2,2-dimethoxy-1-hydroxy)] melamin and tetra-[N-(2,2-dimethoxy-1-hydroxy)] glycouryl.

In one embodiment, the polyamine is a triamine.

In one embodiment, the triamine is melamine.

In one embodiment, the aldehyde is formaldehyde.

In one embodiment, the polyamine-aldehyde pre-condensate is a pre-condensate of melamine-formaldehyde, which can be formed of methylolated melamine formed by the reaction of melamine and formaldehyde, in a manner known per se. Methylolated melamine may also be partially methoxymethylated by the action of methanol on the methylolated melamine.

Milk proteins generally are mixtures of casein and whey proteins, in various proportions, depending on the source of the milk. Milk protein co-precipitates contain both kinds of proteins.

Whey is the liquid left over when milk is coagulated during the process of cheese production, and contains everything that is soluble from milk after the pH is dropped to 4.6 during the coagulation process. The term “whey protein” refers to the proteins contained in the whey. Whey protein is generally understood to include the globular proteins β-lactoglobulin and β-lactalbumin.

Casein is a family of related phosphoproteins (αS1, αS2, β, κ). Casein contains a high number of proline amino acids which hinder the formation of common secondary structural motifs of proteins. There are also no disulfide bridges. As a result, it has relatively little tertiary structure. It is relatively hydrophobic, making it poorly soluble in water.

In one embodiment, the milk protein or milk protein derivative is selected from the group consisting of casein, caseinate salts such as sodium caseinate or calcium caseinate, hydrolysed casein and combinations thereof.

Cross-linking plays an important function in determining the quality of the shell, and therefore the stability and performance of microcapsules. It might be expected that replacing a polyol such as resorcinol, or a diamine such as urea, with a milk protein or a milk protein derivative will affect the microcapsule stability and performance. Nevertheless, the applicant surprisingly found that microcapsules with good stability and performance could be formed.

Particularly stable and performant microcapsules could be obtained by selecting the weight ratio of milk protein or milk protein derivative, e.g. casein, to amino-aldehyde pre-condensate, such as melamine formaldehyde pre-condensate. Hence, in a particular embodiment the of the invention, the ratio of amino-aldehyde pre-condensate to milk protein or milk protein derivative is from about 0.1 to about 1.0, and more particularly about 0.2 to 0.6, even more particularly, about 0.4.

### Polymeric Stabilizer

During the preparation of encapsulated compositions it is conventional to employ a polymeric stabilizer. Polymeric stabilizers act as colloid stabilizers and are employed to stabilize the oil-water interface during microcapsule formation. The polymeric stabilizer functions in several ways: It ensures that stable oil-in-water emulsions are formed allowing migration of shell-forming materials, e.g. pre-condensate and milk protein or milk protein derivative to the oil-water interface; and it functions essentially as a template around which poly-condensation and cross-linking reactions can take place to form the encapsulating cross-linked resin shells. Colloid stabilizers also prevent the formed microcapsules from agglomerating.

In one embodiment, the polymeric stabilizer is an anionic polyelectrolyte.

Particular examples of suitable polymeric stabilizers include acrylic copolymers bearing sulfonate groups, such as those available commercially under the trade mark LUPASOL (ex BASF), such as LUPASOL PA 140 or LUPASOL VFR; copolymers of acrylamide and acrylic acid, copolymers of alkyl acrylates and N-vinylpyrrolidone, such as those available under the trade mark Luviskol (e.g. LUVISKOL K 15, K 30 or K 90 ex BASF); sodium polycarboxylates (ex Polyscience Inc.) or sodium poly(styrene sulfonate) (ex Polyscience Inc.); vinyl and methyl vinyl ether-maleic anhydride copolymers (e.g. AGRIMER™ VEMA™ AN, ex ISP), and ethylene, isobutylene or styrene-maleic anhydride copolymers (e.g. ZEMAC™). Optionally, the polymeric stabilizer is an ethylene, isobutylene or styrene-maleic anhydride copolymer.

In one embodiment, from about 75 wt % to about 100 wt % of the resin comprises
from about 70 wt % to about 95 wt %, preferably from about 80 wt % to about 95 wt %, of terpolymer; and
from about 5 wt % to about 30 wt %, preferably from 5 wt % to about 15 wt %, of polymeric stabilizer.

In one embodiment, the terpolymer comprises
(a) from about 5 wt % to about 25 wt %, preferably from about 10 wt % to about 20 wt % of moieties derived from at least one polyamine,
(b) from about 50 wt % to about 90 wt %, preferably from about 60 wt % to about 80 wt % of moieties derived from a milk protein or a milk protein derivative,
(c) from about 5 wt % to about 25 wt %, preferably from about 10 wt % to about 20 wt % of moieties derived from the group consisting of alkylene and alkylenoxy moieties having 1 to 6 methylene units, preferably 1 to 4 methylene units and most preferably 1 methylene unit.

### Diamine and/or Aromatic Polyol

The encapsulated compositions according to the invention can be employed as an alternative to polyol or diamine-based microcapsules described in WO 2008/098387A1, WO 2016/207180A1 and WO 2017/001672A1A1.

In one embodiment, the encapsulated compositions of the present invention are resorcinol-free, to avoid discolouration issues.

However, the use of small amounts of urea or resorcinol, or other aromatic polyols as more fully described below, as a cross-linker is not excluded in the practice of the present invention. In the case of resorcinol, the levels used should not be so high as to create discolouration issues.

In one embodiment, therefore, the terpolymer of the present invention further comprises moieties derived from at least one diamine or at least one aromatic polyol.

Examples of suitable aromatic polyols include, but are not limited to phenol, 3,5-dihydroxy toluene, bisphenol A, resorcinol, hydroquinone, xylenol, polyhydroxy naphthalene and polyphenols produced by the degradation of cellulose and humic acids. A particularly suitable polyol is resorcinol.

Examples of suitable diamines include but are not limited to urea, 6-substituted 2,4-diamino-1,3,5-triazine and benzoguanidine.

Suitable diamines are urea and 6-substituted-2,4, diamine-1,3,5-triazine. A particularly suitable diamine is urea.

In one embodiment, the terpolymer comprises from about 10 wt % to about 20 wt % of moieties derived from at least one diamine or at least one polyol.

In an encapsulated composition of the present invention, the microcapsule shells may be formed of a single layer, bi-layer or multi-layer, that is, three or more layers. The layers may be compositionally the same, that is, the amino-aldehyde pre-condensate and milk protein or milk protein derivative employed may be the same, or they may be different. Optionally, each layer may be homogeneous or gradual.

Encapsulated compositions of the present invention are prepared in the form of an aqueous slurry, having typically 20 to 50% solids content, and more typically 30 to 45% solid content, wherein the term “solids content” refers to the combined weight of the microcapsule core and shell material expressed as a percentage of the total weight of the slurry.

In order to offer an optimal balance between stability, deposition on substrate and performance, the volume median size (d50) of the microcapsules, measured by laser diffraction, can be from 1 to 60 μm, preferably from 5 to 30 μm, most preferably from 10 to 20 μm. Microcapsules having diameters smaller than 5 μm show large surface to volume ratios and are therefore more prone to leaching, whereas, as the number of microcapsule decreases with increasing diameter, too large microcapsules may not be numerous enough to provide noticeable benefits. Furthermore, large microcapsules may be visible in the product or may visibly stain the substrate.

The slurry may contain formulation aids, such as formaldehyde scavengers, preservatives and stabilizing and viscosity control hydrocolloids.

Typical formaldehyde scavengers comprise compounds capable of binding free formaldehyde in aqueous media, such as sodium sulfite, melamine, glycine, ethylene urea and carbohydrazine. Optionally, the formaldehyde scavenger is ethylene urea.

Typically, the slurries comprise anti-microbial preservatives, which are well known in the art. Examples of suitable preservatives are phenoxy ethanol, caprylyl glycol and combinations thereof.

Suspending aid, such as a hydrocolloid suspending aid assist in the stable physical dispersion of the microcapsules and prevent any creaming or coalescence. Any additional adjuvants conventional in the art may also be added during further-processing.

The encapsulated compositions of the present invention are further characterized in that microcapsules have a nominal shell to core mass ratio in the range from 0.1 to 30%, preferably from 1 to 25% and most preferably from 10 to 20%.

### Method

The invention can be further understood with reference to a method of preparing the encapsulated composition.

Generally, encapsulated compositions may be prepared by first forming an oil-in-water emulsion consisting of benefit agent-containing oil droplets dispersed in an aqueous continuous phase. Thereafter, amino-aldehyde pre-condensate is caused to undergo a poly-condensation reaction to form an encapsulating resin shell around the benefit agent-containing droplets, which resin is cross-linked with a milk protein or milk protein derivative.

In a further aspect, therefore, it is provided a method of making an encapsulated composition as defined herein. The method comprises the steps of:
a) Emulsifying an oil phase comprising at least one benefit agent with an aqueous phase comprising a polymeric stabilizer in the presence of a polyamine-aldehyde pre-condensate to form an emulsion of oil droplets in the aqueous phase;
b) Dissolving or dispersing a milk protein or a milk protein derivative in the emulsion of step a);
c) Dissolving or dispersing a second portion of a polyamine-aldehyde pre-condensate in the composition resulting from step b);
d) Causing the polyamine-aldehyde pre-condensate and the milk protein or milk protein derivative to form a shell at the oil-water interface of the emulsified oil droplets, thereby forming a slurry of microcapsules.

The benefit agent, polymeric stabilizer, polyamine-aldehyde pre-condensate and milk protein or a milk protein derivative are as defined herein.

After formation of the microcapsules, the encapsulated composition is usually cooled to room temperature. Before, during or after cooling, the encapsulated composition may be further processed. Further processing may include treatment of the composition with anti-microbial preservatives and formaldehyde scavengers, as defined herein. Further processing may also include the addition of a suspending aid, such as a hydrocolloid suspending aid to assist in the stable physical dispersion of the microcapsules and prevent any creaming or coalescence. Any additional adjuvants conventional in the art may also be added during further-processing.

Oil-in-water emulsions have the advantage of providing a plurality of droplets that may be used as template for shell formation, wherein the shell is built around each of these droplets. Additionally, the droplet size distribution may be controlled in emulsions, by controlling the conditions of emulsifications, such as stirring speed and stirrer geometry. As a result, a plurality of microcapsules is obtained with controlled average size and size distribution, wherein the oil phase is encapsulated and forms thereby the core of the microcapsules.

The appropriate stirring speed and geometry of the mixer can be selected in order to obtain the desired average droplet size and droplet size distribution. It is a characteristic of the present invention that the polymeric stabilizer has sufficient interfacial activity and is able to promote the formation of dispersed oil droplets with desirable droplet size.

In a process according to the present invention, a 1.5 liter vessel equipped with a turbine, or a cross-beam stirrer with pitched beam, such as a Mig stirrer, and having a stirrer diameter to reactor diameter of 0.6 to 0.8 may be used. Microcapsules formed in such reactor may have a volume median size (d50) of 30 microns or less, at a stirring speed from about 100 to about 1200 rpm. The person skilled in the art will understand that such stirring conditions may change depending on the size of the reactor and of the batch size, on the exact geometry of the stirrer on the ratio of the diameter of the stirrer to the diameter of the reactor diameter ratios.

In particular, the method comprises the steps of:
(i) mixing and dissolving a polymeric stabilizer in water under moderate shear;
(ii) adjusting the temperature to about 30° C., and more particularly 35±2° C. and the pH to 4.6±2 before adding the amino-aldehyde pre-condensate, benefit agent and, optionally the diamine or polyol;
(iii) emulsifying the mixture, whereby the stirring speed and the geometry of the mixer is selected to obtain a desired average droplet size range and droplet size distribution;
(iv) raising the temperature to an elevated temperature above about 70° C., more particularly above about 75° C., (e.g. to 75° C.±1° C.) over a time period of about 1 hour, and more particularly 90 minutes;
(v) adding a milk protein or a milk protein derivative, while the mixture is still at the elevated temperature (e.g. 75° C.);
(vi) Adding a second portion of amino-aldehyde pre-condensate while maintaining the reaction at this elevated temperature for a sufficient period of time (e.g. about 1 hour) to effect poly-condensation and cross-linking reactions, thereby to form microcapsules
(vii) optionally, adding a formaldehyde scavenger, while the mixture is still at the elevated temperature (e.g. 75° C.), before cooling the mixture to room temperature; and
(viii) optionally adding preservatives.

The method according to the present invention may comprise the additional step of drying the microcapsules, in order to obtain a microcapsule power.

Optionally, additional materials may be added to this powder. Suitable additional materials are, for example, carrier materials, such as salts, silicates, clays and carbohydrates, fire proofing materials; functional materials, such as fragrance ingredients, cosmetic ingredients, biologically active ingredients, and substrate enhancers; additional encapsulating materials, such as polysaccharides, proteins, alkoxysilanes, synthetic polymers and copolymers, surfactants and waxes.

Drying methods such as spray-drying, spray-coating, belt and drum drying may be employed. These methods are well known to the art.

In particular, the drying process may be accompanied by an additional encapsulation process, wherein a functional material is entrapped in an additional encapsulating material. For example, the slurry to be dried may comprise, additionally to the core-shell microcapsules obtained in the process according to the present invention, at least one non-encapsulated functional material and at least one water-soluble encapsulating material, so that the functional material, that is not encapsulated in the core-shell microcapsule, is entrapped in the water-soluble encapsulating material during drying. Typically, the at least one water-soluble encapsulating material comprises at least one hydrocolloid, such as starch octenyl succinate and gum acacia. The hydrocolloid promotes and stabilizes the dispersion of the non-encapsulated material in the aqueous phase of the slurry, so that, upon drying, a matrix is formed around or coexisting with the core-shell microcapsules.

The benefit agent that is encapsulated in the core-shell microcapsules may comprise a first fragrance, whereas the functional material entrapped in the water-soluble encapsulating material may comprise a second fragrance, wherein the first and second fragrances are identical or different.

Combining at least two encapsulation processes has the advantage of providing different mechanisms for releasing the benefit agent and the functional material, for example a combination of moisture-induced and mechanical stress-induced releases.

The drying step may also be accompanied or followed by mechanical or thermal treatment, such as spheronization, granulation and extrusion.

In accordance with the process of the present invention, if desired, core-shell microcapsules may be further coated with a functional coating. A functional coating may entirely or only partially coat the microcapsule shell. Regardless whether the functional coating is charged or uncharged, its primary purpose is to alter the surface properties of the microcapsule to achieve a desirable effect, such as to enhance the deposition of the microcapsule on a treated surface, such as a fabric, human skin or hair. Functional coatings may be post-coated to already formed microcapsules, or they may be physically incorporated into the microcapsule shell during shell formation. They may be attached to the shell by physical forces, physical interactions, such as hydrogen bonding, ionic interactions, hydrophobic interactions, electron transfer interactions, or they may be covalently bonded to the shell.

The encapsulated composition may be in the form of liquid slurries, powder, granulates, flakes or extrudates. The composition may be used as such, for example as fragrance booster, or in diluted form in a product.

Encapsulated compositions in the form of liquid slurries may comprise from 10% to 50%, more particularly from 25% to 45%, of core-shell microcapsules.

Encapsulated compositions in solid form may comprise from 1 to 100% of core-shell microcapsules. However, depending on the application or on the nature of the functional material, it may be preferable to limit or, on the contrary, to maximize the level of core-shell microcapsules in the solid form. For example, a limitation of the level of the core-shell microcapsules in the solid may be particularly desired if the encapsulated material is flammable, reactive, pungent or expensive.

Hence, the optimal level of encapsulated fragrance ingredients in a solid composition may be less than 50%, more particularly less than 35% and still more particularly less than 20%, or even less than 15%, depending on the flammability of such fragrance ingredients and the associated explosion risks.

The encapsulated fragrance may be diluted in a carrier material mentioned herein above.

### Consumer Product

The present invention also relates to a consumer product comprising an encapsulated composition as described hereinabove. The consumer product may be selected from the group consisting of household (home) care, personal care, fabric care and pet care products.

Suitable home care products include hard surface cleaners, heavy duty detergents and detergent powders, air care compositions.

Suitable personal care products include cleansing compositions (such as shampoos, bath and shower gels, liquid soaps, soap bars), conditioning compositions (such as hair care conditioners), bath and shower lotions, oral care compositions, deodorant compositions, antiperspirant compositions, skin care products

Suitable fabric care compositions include laundry care detergents, laundry care conditioners, fabric refreshers, scent boosters.

Encapsulated compositions according to the present invention are particularly useful when employed as perfume delivery vehicles in consumer goods that require, for delivering optimal perfumery benefits, that the microcapsules adhere well to a substrate on which they are applied. Such consumer goods include hair shampoos and conditioners, as well as textile-treatment products, such as laundry detergents and conditioners.

The encapsulated composition of the present invention, presented in the form of a slurry of microcapsules suspended in an aqueous suspending medium may be incorporated as such in a consumer product base. If desired, however, the slurry may be dried to present the encapsulated composition in dry powder form. Drying of a slurry of microcapsules is conventional, and may be carried out according techniques known in the art, such as spray-drying, evaporation, lyophilization or use of a desiccant. Typically, as is conventional in the art, dried microcapsules will be dispersed or suspended in a suitable powder, such as powdered silica, which can act as a bulking agent or flow aid. Such suitable powder may be added to the encapsulated composition before, during or after the drying step.

Yet another aspect of the present invention relates to the use of an encapsulated composition or consumer product as described hereinabove to improve the perception or enhance the performance of the benefit agent in the consumer product.

The present invention is further illustrated by means of the following non-limiting examples:

### EXAMPLES

The solid content of each of the slurries was measured by using a thermobalance operating at 120° C. The solid content, expressed as weight percentage of the initial slurry deposited on the balance was taken at the point where the drying-induced rate of weight change had dropped below 0.1%/min. The ratio of the measured solid content to the theoretical solid content calculated based on the weight of perfume and encapsulating materials involved is taken as a measurement of encapsulation yield, expressed in %.

Viscosity was measured at 25° C. at 21 s⁻¹ with a RheolabQC rheometer from Anton Paar.

Formaldehyde residues were measured using LC-UV derivatization, C18 column. The results are expressed as an average of four measurements.

### Example 1: Capsule 1, Melamine-Caseinate-Formaldehyde Cross-Polymer

In a 1.5 L reactor, 61 grams of a 10% ZeMac solution is added followed by 293.5 grams of water. The pH is increased using a 30% NaOH solution to reach 4.6+/−0.2. The overhead stirrer speed is set at 650 RPM while adding 330.6 grams of fragrance. The temperature is set at 35° C. before adding 13.1 grams of Luracoll (70% melanime-formaldehyde pre-condensate). The pH at this stage is around 4.9.

After 30 minutes at 35° C., 30 minutes of ramping up to 75° C. and 1 h 30 minutes at 75° C., the stirring is increased to 800 RPM prior to adding Caseinate solution to the medium (202.7 grams, 15% Sodium Caseinate, viscosity=1400 mPa·s @ 21 s⁻¹, pH 5.6, prepared from purified Casein, Aldrich and 30% NaOH). This brings the pH at 5.3 in the reactor.

After 30 minutes, 6 grams of Luracoll are added and stirring is continued for another hour at 75° C. Subsequently, ethylene urea is added as a scavenger of free formaldehyde. The slurry is cooled down to 25° C. in 1 hour. Preservatives phenoxy ethanol (4 g) and caprylyl glycol (4 g) are added. The slurry is sieved (212 microns) prior to characterisation and analyses.

Formaldehyde residue was 6 ppm.

The measured biodegradability of the microcapsules was measured according to OECD Method 301F after 60 Days as 70%.

Therefore, the melamine-caseinate-formaldehyde cross-polymer passes the test for biodegradability.

### Example 2: Control 1, Melamine-Formaldehyde Cross-Polymer, No Caseinate

The procedure is the same as Example 1, except that no Caseinate is added. The 202.7 grams of caseinate solution in Example 1 is replaced by water.

### Example 3: Control 2, Melamine-Formaldehyde Cross-Polymer, Ungrafted Caseinate

The procedure is the same as Example 1, except that Caseinate is added after the process of polycondensation, at 25° C., formaldehyde scavenging by ethylene-urea and the preservatives addition.

### Example 4: Benchmark, Melamine-Resorcinol-Formaldehyde Cross-Polymer

Microcapsules employing a resorcinol cross-linker were prepared according to the procedure described in WO 2016/207180A1.

### Example 5: Comparison of Physical Properties of the Microcapsules in Examples 1-3

The solid content, volume median size (d50) and viscosity of the slurries obtained in Examples 1 to 3 are shown in Table 1.

| Example | Solid content (%) | d50 (μm) | Viscosity (mPa · s) |
|---|---|---|---|
| 1 | 39 | 14.7 | 200-300 |
| 2 | 9 | 30.4 | 100 |
| 3 | 16 | 21.6 | 2300 |

It can be observed that the encapsulation is efficient only when the caseinate is added to the melamine-formaledehyde pre-condensate under favorable conditions of condensation, during the process of curing (Example 1), the solid content of the microcapsules being below the specification of 20% in case no caseinate was employed (Example 2) or when caseinate was added after condensation has taken place (Example 3).

It can also be observed that the size of microcapsules satisfies the preferred specification requirements of volume median size between 10-20 μm only in case of Example 1.

In the absence of caseinate (Example 2), the viscosity of the slurry is very low (about 100 mPa, Example 2), whereas when caseinate is added post-condensation (Example 3), the viscosity becomes very high (about 2300 mPa). However, addition of caseinate in conditions favourable for it to participate in condensation leads to a viscosity in the range between 150 mPa to 500 mPa, providing evidence that the caseinate participates in a coupling reaction with the melamin-formaldehyde pre-condensate and no or limited ungrafted caseinate remained in the aqueous phase.

### Example 6: Comparison of Olfactive Performance

The olfactive performance of the microcapsules was assessed by a panel of 4 experts who rated the odor intensity on a scale of 1-5 (1=barely noticeable, 2=weak, 3=medium, 4=strong and 5=very strong). When relevant, qualitative comments on the perceived odor direction were recorded.

For application in laundry care using a washing machine, the samples were evaluated in an unperfumed commercial proprietary fabric care softener. The aforementioned microcapsule slurries were added to a fabric care conditioner composition under gentle stirring with a paddle mixer, so that the level of slurry in the fabric care conditioner base was 0.6% referred to the total weight of the fabric care softener base. 35 g of fabric care conditioner was put in a front-loaded European wash machine containing 720 g of terry toweling and operating with a total volume of 15 l water. The “out-of-the-wash machine” odor intensity was assessed on wet toweling within 5 min after having removed the toweling from the machine. The pre-rub olfactive evaluation was performed after drying the toweling for 24 h at room temperature. The post-rub evaluation was performed by gently rubbing one part of the toweling. The performance on terry toweling of freshly prepared and aged microcapsules is shown in Table 2.

TABLE 2
Olfactive performance of microcapsules of
Examples 1 and 4 on terry toweling (washing machine)

| | Intensity | Pre-rub | Post-rub |
|---|---|---|---|
| Example | on wet | intensity on dry | intensity on dry |
| 1 | 2.5 | 2 | 2.5 |
| 4 | 1 | 2.8 | 3.5 |

For application in hand washing, the following protocol was followed:
Wash load: 2 pieces of 100% cotton terry towels. (Approximately 70-80 g, 30 cm×30 cm)
Water volume: 1 L
Water details: Standard tap water, with 57 ppm water hardness
1. Shake fabric softener composition (at 4 weeks after preparation).
2. Add 5 ml fabric softener into a bowl containing 1 L of water.
3. Stir solution for 30 seconds to ensure product is evenly dispersed in the water.
4. Soak the towels for 10 minutes. Wring the towel. Evaluate the “intensity on wet”.
5. Drying indoors-Hang the towel on a rack for over 24 hours to dry inside a drying room with help of a fan (temperature 25° C., with no air-condition) during the drying process.
6. Assess the towel for “intensity on dry” for 24 hours (pre-rub & post-rub).

The performance of aged microcapsules on terry toweling is shown in Table 3.

TABLE 3
Olfactive performance of microcapsules of Examples
1 and 4 on terry toweling (hand washing after 4 weeks, 40° C.)

| | Intensity | Pre-rub | Post-rub |
|---|---|---|---|
| Example | on wet | intensity on dry | intensity on dry |
| 1 | 1.5 | 1.75 | 1.25 |
| 4 | 1 | 1 | 1.17 |

Data in tables 2 and 3 shows that the biodegradable microcapsules of the present invention perform comparably with the benchmark aminoplast microcapsules of the prior art comprising resorcinol as a cross-linker.

1. An encapsulated composition comprising at least one core-shell microcapsule, wherein the at least one core-shell microcapsule comprises a core comprising at least one benefit agent and a shell surrounding the core, wherein the shell comprises a network of cross-linked resin, wherein the resin comprises a terpolymer and a polymeric stabilizer, wherein the terpolymer comprises:
(a) moieties derived from at least one polyamine,
(b) moieties derived from a milk protein or a milk protein derivative,
(c) moieties derived from the group consisting of alkylene and alkylenoxy moieties having 1 to 6 methylene units.

2. The encapsulated composition according to claim 1, wherein from about 75 wt % to about 100 wt % of the resin comprises:
from about 70 wt % to about 95 wt % of the terpolymer; and
from about 5 wt % to about 30 wt % of the polymeric stabilizer.

3. The encapsulated composition according to claim 1, wherein the terpolymer comprises:
(a) from about 5 wt % to about 25 wt % of moieties derived from at least one polyamine,
(b) from about 50 wt % to about 90 wt % of moieties derived from a milk protein or a milk protein derivative,
(c) from about 5 wt % to about 25 wt % of moieties derived from the group consisting of alkylene and alkylenoxy moieties having 1 to 6 methylene units.

4. The encapsulated composition according to claim 1, wherein the terpolymer comprising the moieties a), b) and c) is a condensation product of a polyamine-aldehyde pre-condensate cross-linked with moieties derived from a milk protein or a milk protein derivative.

5. The encapsulated composition according to claim 1, wherein:
i) the polyamine is a triamine; and/or
ii) the aldehyde is formaldehyde.

6. The encapsulated composition according to claim 1, wherein the milk protein or milk protein derivative is selected from the group consisting of casein, caseinate salts, hydrolysed casein and combinations thereof.

7. The encapsulated composition according to claim 1, wherein the polymeric stabilizer is an anionic polyelectrolyte.

8. The encapsulated composition according to claim 1, wherein the terpolymer further comprises moieties derived from at least one diamine or at least one aromatic polyol.

9. The encapsulated composition according to claim 8, wherein the terpolymer comprises from about 10 wt % to about 20 wt % of moieties derived from at least one diamine or at least one polyol.

10. The encapsulated composition according to claim 8, wherein:
i) the at least one diamine is selected from the group consisting of urea and 6-substituted 2,4-diamino-1,3,5-triazin; and
ii) the at least one polyol is selected from the group consisting of phenol, 3,5-dihydroxy toluene, Bisphenol A, resorcinol, hydroquinone, xylenol, polyhydroxy naphthalene and polyphenols produced by the degradation of cellulose and humic acids.

11. The encapsulated composition according to claim 1, wherein the microcapsule is a formed of a single layer, bi-layer or multi-layer.

12. The encapsulated composition according to claim 1, wherein the benefit agent is selected from the group consisting of perfume or fragrance ingredients and cosmetic ingredients.

13. A method for making an encapsulated composition according to claim 1, comprising the steps of:
a) Emulsifying an oil phase comprising at least one benefit agent with an aqueous phase comprising a polymeric stabilizer in the presence of a polyamine-aldehyde pre-condensate to form an emulsion of oil droplets in the aqueous phase;
b) Dissolving or dispersing a milk protein or a milk protein derivative in the emulsion of step a);
c) Dissolving or dispersing a second portion of a polyamine-aldehyde pre-condensate in the composition resulting from step b);
d) Causing the polyamine-aldehyde pre-condensate and the milk protein or milk protein derivative to form a shell at the oil-water interface of the emulsified oil droplets, thereby forming a slurry of microcapsules.

14. A consumer product comprising the encapsulated composition according to claim 1.

15. A method of improving the perception of, or enhancing the performance of the benefit agent in a consumer product, comprising using the encapsulated composition of claim 1 within the consumer product.

16. The consumer product according to claim 14, selected from the group consisting of: a personal care product, a fabric care product, a home care product and a pet care product.

17. The method of claim 15, wherein the consumer product is selected from the group consisting of: a personal care product, a fabric care product, a home care product and a pet care product.

18. The encapsulated composition according to claim 1, wherein the polyamine is melamine.

19. The encapsulated composition according to claim 7, wherein the polymeric stabilizer is selected from the group consisting of: acrylic copolymers bearing sulfonate groups, copolymers of acrylamide and acrylic acid, copolymers of alkyl acrylates and N-vinylpyrrolidone, sodium polycarboxylates or sodium poly(styrene sulfonate), vinyl and methyl vinyl ether-maleic anhydride copolymers and ethylene, isobutylene or styrene-maleic anhydride copolymers.
